(19) United States
(12) Patent Application Publication
(10) Pub. No.: US 2004/0045558 A1
(43) Pub. Date: Mar. 11, 2004

(54) Earplug and method of manufacturing an earplug

(76) Inventors: Duncan Taylor, Carmel, IN (US); Bob Klun, Zionsville, IN (US)

Correspondence Address: CANTOR COLBURN, LLP, 55 GRIFFIN ROAD SOUTH, BLOOMFIELD, CT, 06002

(21) Appl. No.: 10/236,595

(22) Filed: Sep. 6, 2002

(51) Int. Cl. A61F 11/00

(52) U.S. Cl. 128/864

(57) ABSTRACT

An earplug and a method of manufacture thereof is provided, the earplug includes a foam having an insertion portion for being inserted into an ear canal of a user and a skirt extending from the insertion portion. The skirt includes a receptacle formed at an interior thereof. The earplug further includes a molded stem having a bonding portion which is received in the receptacle and a handle portion which extends from the receptacle. The handle portion facilitates handling of the earplug during manufacture and use thereof. The skirt extends around the bonding portion of the stem and the bonding portion and the foam are adhesively bonded in the receptacle.

### BACKGROUND OF INVENTION

(1) Field of Invention

The present invention relates generally to hearing protective devices, and more particularly to an earplug including an injection molded stem adhesively bonded to a foam.

(2) Description of Related Art

The use of hearing protective and noise attenuating devices is well known, and various types of devices are available including semi-aural devices and earplugs. Foam components are well known for use with these devices, providing both sound attenuation and enhanced comfort for the wearer.

Earplugs are generally utilized for continuous use over longer periods of time. Foam earplugs composed of resilient polymer foam materials such as foam rubber or polyurethane are well known in the art. Slow recovery foam earplugs, such as those disclosed in U.S. Reissue. No. 29,487 to Gardner, Jr., provide comfort and have also been shown to deliver high in-field noise protection at all frequencies. U.S. Pat. No. 5,203,352 to Gardner, Jr. also discloses a hearing protective earplug comprising a polymeric foam.

Such earplugs may further include a cord that attaches a pair of plugs and extends therebetween. Conventional cords are commonly designed so that the cord may be permanently attached to the earplugs and more specifically, each end of the cord may be permanently attached to ends of stem portions of the respective earplugs.

The sound-attenuating (acoustical) component of earplugs has been made both of foam, as described above, and of flexible, rubber-like materials. The rubber-like materials are advantageous because they allow direct and easy insertion of the earplug into the wearer's ear. However, such earplugs and devices typically become uncomfortable over long periods of use. In this respect, foam earplugs are preferred for enhanced comfort.

Foam earplugs of the roll-down type are known in the art. These earplugs are generally cylindrical in shape and include a body composed of a resilient foam material. In use, a wearer rolls the body to compress the earplug, thus reducing a cross-sectional area thereof. In this compressed state, one end of the earplug is inserted into the ear canal while the opposite end serves as a handle to aid insertion. Once inserted in the ear canal, the plug expands towards its original dimensions, filling a portion of the ear canal, and providing a comfortable fit and suitable attenuation properties.

However, foam earplugs of the roll-down type are often difficult to fully insert due to the pliable nature of such plugs. That is, even in the rolled down state, the plug still may not provide adequate rigidity to insert the body of the plug a sufficient distance into the ear canal. Insufficient insertion results in inadequate attenuation and is thus undesirable.

To remedy the insertion deficiencies of the roll-down type earplugs, foam earplugs have been developed to include rigid or semi-rigid stem components. Such earplugs include a resilient foam body and a rigid or semi-rigid stem portion embedded in the foam portion. The stem portion may be embedded entirely in the foam portion or may include a first end embedded in or attached to the foam portion and a second end extending from the foam portion. The stem portion is intended to provide a degree of rigidity to the earplug to aid the wearer in inserting the earplug into the ear canal. That is, the rigidity of the stem portion enables the wearer to push the earplug into the ear canal to a proper insertion depth thus allowing the earplug to provide adequate attenuation. The stem portion also often includes a degree of pliability to ensure comfort of the earplug once inserted into the ear canal.

A strong attachment of the stem component to the foam is critical to ensure functionality of the earplug. Typically, the stem is mechanically bonded to the foam during the earplug manufacturing process. First, the stem is manufactured and then placed in a mold as an insert. The foam portion is then formed in the mold around a portion of the stem such that the foam mechanically bonds to the stem insert. The result is an earplug including a foam bonded with a stem, the stem extending from the foam. Alternatively, the entire stem may be placed in the mold and the foam allowed to form entirely around the stem and bond thereto, thus encapsulating the stem. In this case, the resulting earplug includes a foam outer body and a stem portion embedded at an interior of the body.

Mechanically bonding the foam and stem as described is a difficult and often inconsistent process. For example, proper formation of the foam and proper bonding of the foam to the stem require adequate venting of the mold in which the foam is formed about the stem. Various attempts have been made at properly venting the molds. For example, a porous stem is disclosed in U.S. Pat. No. 5,799,658 to Falco. Therein, individual porous stems are mechanically bonded to the foam during manufacture by controlled penetration of the foam into the pores of the stem. Air entrapped in the mold is permitted to vent during the process through the pores. However, the individual porous stems are relatively expensive and somewhat difficult to manufacture and proper venting is often not attained. In another example, venting means may be added to the exterior of stems or to the interior of molds to create venting channels or pathways between the stem and mold which lead from an interior of the mold to an exterior. Again, such additions to stems and molds are costly and their effectiveness in properly venting the mold is often found to be insufficient.

Non-automated, manual gluing of the foam and stem to form the earplug has also been attempted. However, an insufficient bond is often formed between the foam and stem when utilizing this process. Further, such process is messy, labor intensive, time consuming, and costly, thus not presenting a suitable means for automated mass production of stem component foam earplugs.

Accordingly, there remains a need for a stemmed foam earplug that is effective in providing hearing protection, yet convenient and comfortable to use, and has a more efficient and lower cost method of manufacture.

### SUMMARY OF INVENTION

The above discussed and other problems and deficiencies of the prior art are overcome or alleviated by the hearing protective device and method of manufacture of the invention.

In one embodiment of the invention, an earplug comprises a foam including an insertion portion for being inserted into an ear canal of a user and a skirt extending from the insertion portion. The skirt includes a receptacle formed at an interior thereof. The earplug further comprises a molded stem including a bonding portion which is received in the receptacle and a handle portion which extends from the receptacle. The handle portion facilitates handling of the earplug during manufacture and use thereof. The skirt extends around the bonding portion of the stem and the bonding portion and the foam are adhesively bonded in the receptacle.

In another exemplary embodiment, an earplug comprises a foam including a rounded insertion portion for being inserted into an ear canal of a user, a cylindrical skirt extending from the insertion portion, a receptacle formed at an interior of the skirt, and ribs extending from the cylindrical skirt to the rounded insertion portion. The earplug further comprises a molded stem including a bonding portion having a flattened surface, a handle portion having contouring to facilitate handling of the earplug during manufacture and use of the earplug, and a collar disposed intermediate the bonding portion and the handle portion. The skirt extends around the bonding portion. The flattened surface of the bonding portion receives a bonding agent to adhesively bond the bonding portion and the foam in the receptacle.

In a further embodiment of the invention, a method of manufacturing an earplug comprises molding a foam to include a rounded portion and a skirt extending from the rounded portion, forming a receptacle at an interior of the skirt, molding a stem to include a bonding portion having a flattened surface and a handle portion having contouring to facilitate gripping the stem, applying a bonding agent to the flattened surface, inserting the bonding portion into the receptacle, and curing the bonding agent to adhesively bond the bonding portion and the foam in the receptacle.

The above discussed and other features and advantages of the present invention will be appreciated and understood by those skilled in the art from the following detailed description and drawings.

### BRIEF DESCRIPTION OF THE DRAWINGS

Referring now to the drawings wherein like elements are numbered alike in the several FIGURES:

FIG. 1 is perspective view of an earplug according to the invention;

FIG. 2 is a side elevational view thereof;

FIG. 3 is another side elevational view thereof;

FIG. 4 is a top plan view thereof;

FIG. 5 is a bottom plan view thereof;

FIG. 6 is a perspective view of a foam of the earplug of FIG. 1;

FIG. 7 is a cross-sectional view of the foam of FIG. 6;

FIG. 8 is a perspective view of a stem of an earplug according to the invention;

FIG. 9 is side elevational view thereof;

FIG. 10 is another side elevational view thereof;

FIG. 11 is a bottom plan view thereof;

FIG. 12 is a top plan view thereof;

FIG. 13 is a cross-sectional view thereof; and

FIG. 14 is a perspective view of the earplug of FIG. 1 with a cord attached thereto.

### DETAILED DESCRIPTION OF INVENTION

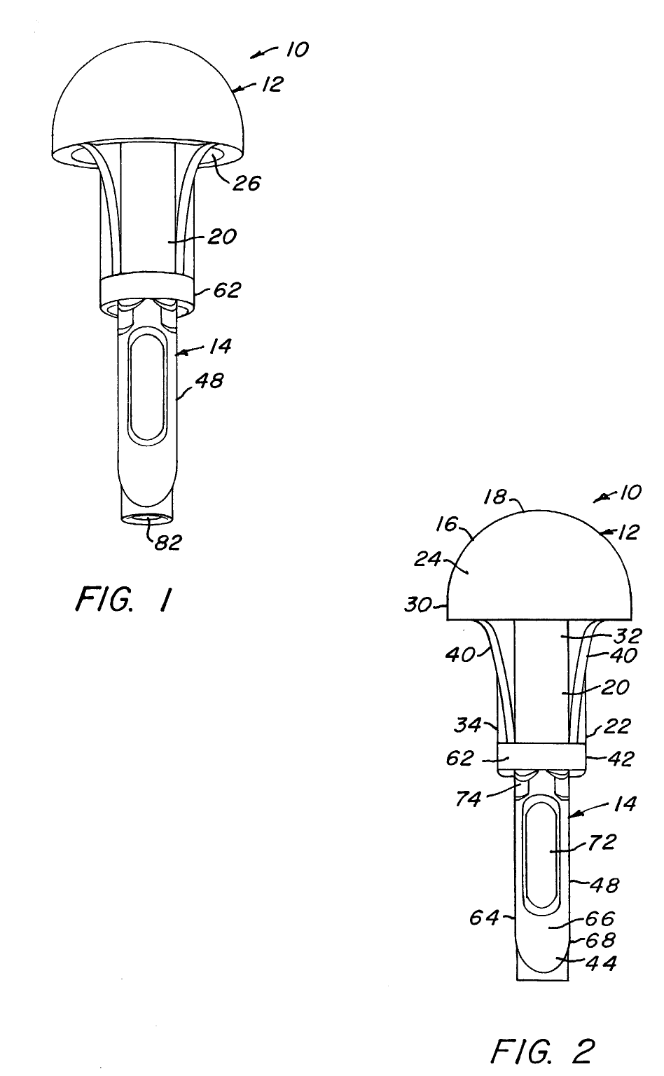

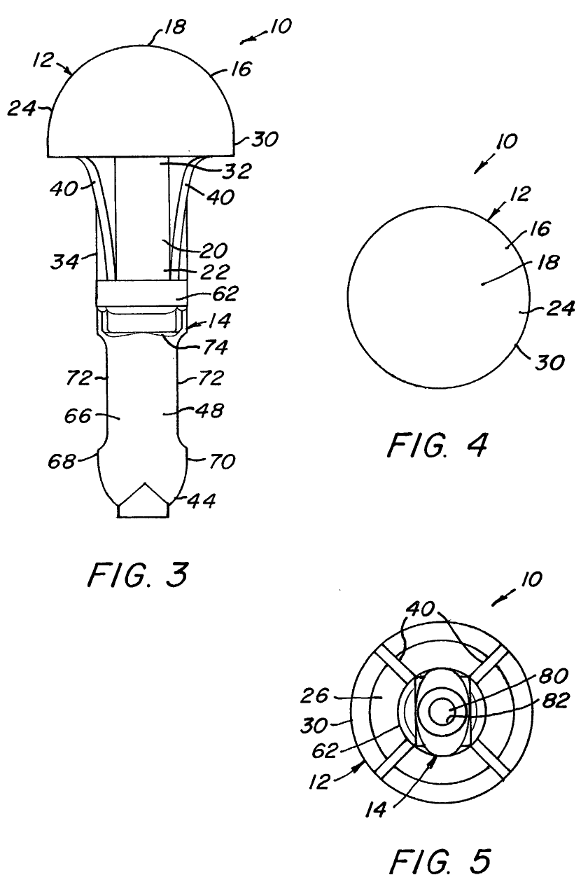

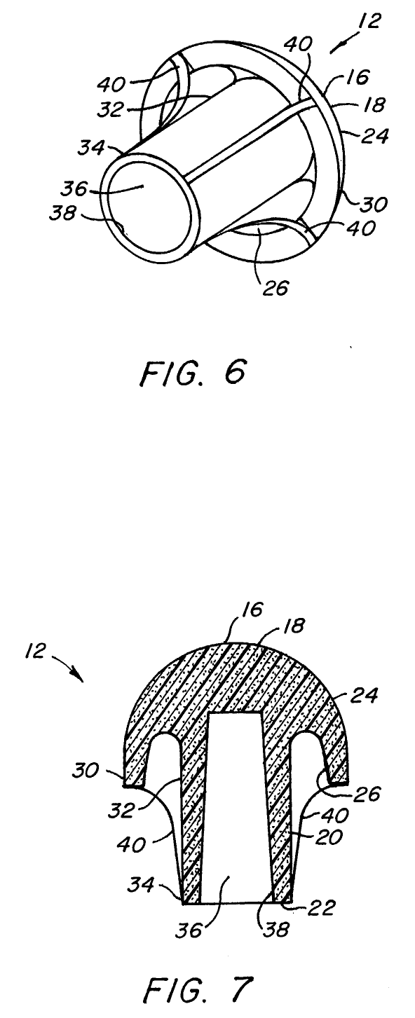

FIGS. 1-5 show various views of an earplug 10 in one embodiment of the invention. The earplug 10 includes a foam 12 and a stem 14. The foam 12 includes a rounded portion 16 at a first end 18 of the foam 12 and a skirt 20 at an opposite second end 22 of the foam 12.

The rounded portion 16 has a substantially semi-hemispherical shape and includes a rounded surface 24 formed proximate the first end 18 of the foam 12 and a concave surface 26 disposed opposite the rounded surface 24. The rounded and concave surfaces 24 and 26, respectively, give the rounded portion 16 a generally crescent shaped axial cross-section (FIG. 7). As such, an edge 30 is formed at an interface between the rounded and concave surfaces 24 and 26, respectively. The edge 30 comprises essentially a flap of the rounded portion 16 is pivotably movable relative to a longitudinal axis of the earplug 10. This feature provides comfort and ensures a proper fit when the earplug 10 is inserted into an ear canal of a user.

The skirt 20 extends from the rounded portion 16 and is generally a cylindrical element having a first skirt end 32 formed integrally with the rounded portion 16 at the concave surface 26 and a second skirt end 34 being free relative to the rounded portion 16. The skirt 20 includes a receptacle 36. The receptacle 36 extends the length of the skirt 20 from the first skirt end 32 to the second skirt end 34 where the receptacle 36 opens to an exterior of the foam 12. At the first skirt end 32, the receptacle 36 exposes a portion of the concave surface 26.

A cross-sectional area of the receptacle 36 is generally circular in shape and is of a consistent dimension from the first skirt end 32 to the second skirt end 34. That is, the receptacle 36 has a substantially cylindrical shape. Of course, the shape of the receptacle 36 may be other than cylindrical. The receptacle 36 may, for example, include a circular or rectilinear cross-sectional area which varies in dimension throughout the length of the receptacle 36. In one such embodiment, the receptacle 36 includes a circular cross-sectional area having a larger dimension proximate the second skirt end 34 and a smaller dimension proximate the first skirt end 32 such that the receptacle 36 tapers toward the first skirt end 32. In another embodiment, the receptacle 36 has a shape including rounded and flat portions and a cross-sectional area which tapers in a direction toward the first skirt end 32. The receptacle 36 may extend past the concave surface 26 into the rounded portion 16 (FIG. 7).

The skirt 20 is generally elongated so as to include an axial length X equal to or greater than an axial length Y of the rounded portion 16.

The foam 12 further includes ribs 40 extending between the rounded portion 16 and the skirt 20. The ribs 40 are essentially flat members formed integrally with the cylindrically shaped skirt 20 and extend radially therefrom to the concave surface 26 of the rounded portion 16. The ribs 40 are fixed to the first skirt end 32 and include a portion extending to the second skirt end 34. In a preferred embodiment, the foam 12 includes four ribs 40 disposed equi-angularly about an outer surface of the skirt 20.

The foam 12 is manufactured in a molding process, as described in more detail herein below. The molding process includes a venting step which releases air entrapped in the mold and allows the foam 12 to properly form therein. The venting step also allows for the ribs 40 to form integrally with the foam 12.

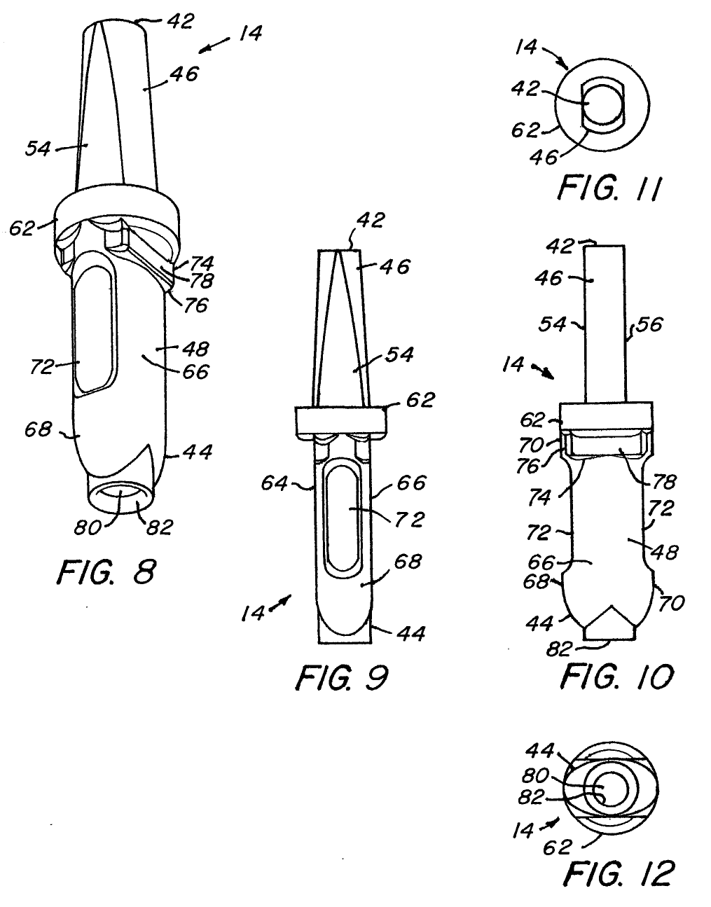

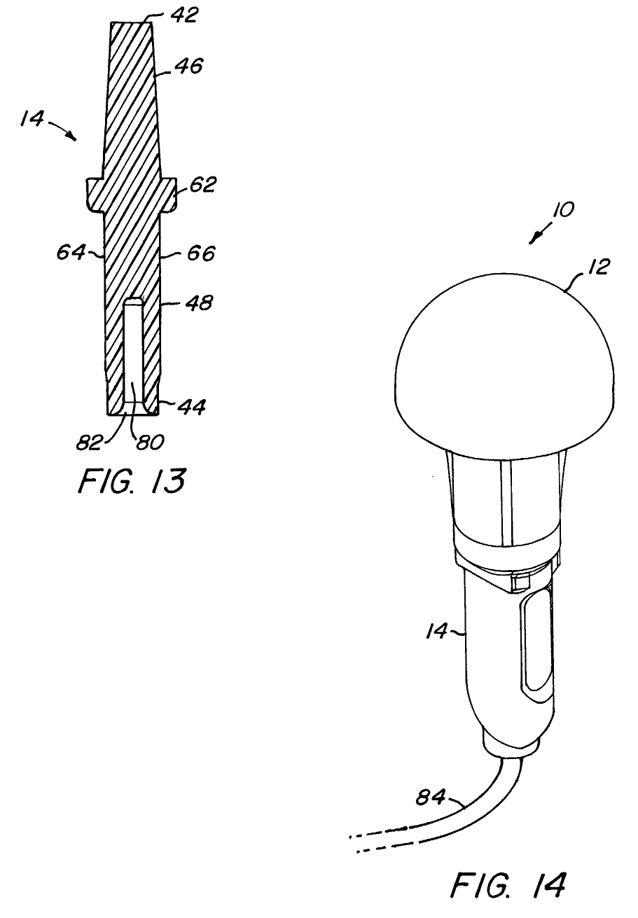

Referring now particularly to FIGS. 8-12, the stem 14 includes a first stem end 42 and an opposing second stem end 44. A bonding portion 46 is formed at the first stem end 42 and a handle portion 48 is formed at the second stem end 44.

The bonding portion 46 is generally a cylindrical element and, in one embodiment, includes a circular cross-sectional area which tapers in a direction toward the first stem end 42, i.e., the circular cross-sectional area is larger proximate the second stem end 44 and gradually becomes smaller along a length of the bonding portion 46 in a direction toward the first stem end 42.

The bonding portion 46 includes a first flattened surface 54 and a second flattened surface 56. The first flattened surface 54 is a planar portion on the surface of the bonding portion 46 which is otherwise generally cylindrical in shape. The first flattened surface 54 extends from the first stem end 42 in a direction toward the second stem end 44. The first flattened surface 54 includes a width W which gradually increases over the extension of the first flattened surface 54, i.e., the width W proximate the first stem end 42 is smaller than the width W proximate the second stem end 44. The second flattened surface 56 is substantially identical to the first flattened surface 54 and is disposed on the bonding portion 46 opposite the first flattened surface 54. As a result of the first and second flattened surfaces 54 and 56, the generally cylindrical shape of the bonding portion 46 includes two substantially flattened sides.

The handle portion 48 extends from the second stem end 44 in a direction toward the first stem end 42 and meets the bonding portion 46 at a point approximately midway between the first and second stem ends 42 and 44. The handle portion 48 includes a collar 62 proximate the bonding portion 46. The collar 62 is a cylindrical element having a cross-sectional area greater than the cross-sectional area of the bonding portion 46. In one embodiment, the collar 62 has a cross-sectional area greater than a cross-sectional area of the remaining portion of the handle portion 48, i.e., the collar circumferentially extends beyond a remaining section of the handle portion 48 as well as beyond the bonding portion 46 such that the stem 14 has a largest cross-sectional area at the collar 62.

The handle portion 48 includes a first side 64 and an identical opposite second side 66. The first and second sides 64 and 66 extend from the second stem end 44 to the collar 62. The handle portion 48 further includes a third side 68 and a fourth side 70, the fourth side 70 being identical to the third side 68 and being disposed opposite the third side 68.

The first and second sides 64 and 66 are generally disposed parallel to one another and the third and fourth sides 68 and 70 are generally parallel to one another. The first and second sides 64 and 66 are generally disposed perpendicular to the third and fourth sides 68 and 70. The third side and fourth sides 68 and 70 are disposed between and substantially connect the first and second sides creating the three-dimensional shape of the handle portion 48 as shown.

The first, second, third, and fourth sides, 64, 66, 68, and 70, are arcuate in shape. More particularly, the first and second sides 64 and 66 comprise a first arcuate surface having a first radius of curvature and the third and fourth sides 68 and 70 comprise a second arcuate surface having a second radius of curvature where the first radius of curvature is greater than second radius of curvature such that an overall width of the first and second sides 64 and 66 is greater than an overall width of the third and fourth sides 68 and 70. The first, second, third, and fourth sides, 64, 66, 68, and 70, combine to form the thin rounded member of the handle portion 48.

The third and fourth sides 68 and 70 each include an indent 72. The indent 72 is a recessed portion formed in the surface of the third and fourth sides 68 and 70. The indents 72 extend longitudinally from the collar 62 towards the second stem end 44 and terminate at a point before the second stem end 44. The indents 72 provide a section on the handle portion 48 of a narrow cross-sectional area relative to elsewhere on the handle portion 48. The indents 72 essentially create a recessed gripping area on the handle portion 48. This gripping area facilitates handling of the stem 14 during molding of the stem and during assembly of the stem and foam. Additionally, the gripping area created by the indents 72 provides enhanced grip to a user when inserting, removing and generally handling the earplug 10. Gripping elements may be integrally formed on the gripping area to provide such enhanced grip. Also, the recessed area created by indents 72 creates a display surface which may be used for displaying characters such as numbers, letters, and/or symbols to a visual observer. Brand names, logos, product information, technical specifications, advertising items, etc. may be formed on the display surface during molding of the stem 14 for visual display purposes.

The first and second sides 64 and 66 each include a footing 74 disposed proximate the collar 62. The footing 74 includes a base 76 extending from the each of the first and second sides 64 and 66. A flat surface 78 is formed atop the base 76. In one embodiment, the footing 74 is flush on one side against the collar 62.

The handle portion 48 of the stem 14 further includes a cavity 80 formed at an interior of the handle portion 48. The cavity 80 is exposed to an exterior of the stem 14 at opening 82 formed at the second stem end 44. The cavity 80 extends axially along a length of the handle portion 48 in a direction toward the first stem end 44. In one embodiment, the cavity 80 terminates at a point approximately midway between the second stem end 44 and the collar 44. The cavity 80 has a substantially circular cross-section which, in one embodiment tapers in a direction toward the collar 62, i.e., the cross-sectional area of the cavity 80 gradually reduces over the length of the cavity in a direction from the second stem end 44 toward the collar 62. The cavity 80 and the opening 82 are particularly designed to receive and retain an earplug cord 84 as shown, for example, in FIG. 14.

The stem 14 is manufactured of a plastic material in a molding process. Particularly, the stem 14 is manufactured in an injection molding process. The foam 12 is manufactured in a molding process separately from the stem 14. In such process, the foam 12 is placed in a mold in liquid form; the foam is allowed to expand, taking the shape of the mold; the mold is properly vented during expansion of the foam 12 to allow air entrapped in the mold to escape the mold; the foam 12 is allowed to cure; and, finally, the foam 12 is ejected from the mold.

The stem 14 is molded into a specific shape to facilitate the molding process there of and the subsequent earplug assembly process. For example, the stem 14 and, particularly, the handle portion 48 provide various surfaces by which injection molding machines and related earplug assembly apparatuses may easily grip the stem 14. Elements of the stem 14 which enable such gripping include, but are not limited to, the following: the collar 62; the first through fourth sides 64, 66, 68, and 70; the indents 72; the footing 74; the flat surface 78; and the relative positioning of the first flattened surface 54 and the second flattened surface 56.

Additionally, the center of gravity of the stem 14 may be located in a position so as to facilitate assembly of the earplug 10. In one embodiment, the center of gravity of the stem 14 is located in a position such that if the stem 14 is dropped during a stage of the assembly process, the second stem end 44 is consistently displaced during the dropping so as to be beneath the first stem end 42. Alternatively, of course, the center of gravity of the stem 14 may be placed such that the first stem end 42 is positioned beneath the second stem end 44 during the dropping of the stem 14. Typically, the third and fourth handle portion sides 68 and 70 and the flat surface 78 control axially rolling of the stem 14. The invention additionally contemplates that the center of gravity of the entire earplug 10, including the stem 14 and foam 12, may be selected so as to facilitate manufacture, assembly, and/or usage thereof.

The earplug 10 is assembled by adhesively bonding the foam 12 to the stem 14. Particularly, an adhesive is placed on one of the first or second flattened surfaces 54, 56 of the bonding portion 46. The bonding portion 46 is then inserted through the opening 38 into the receptacle 36 of the skirt 20. The adhesive agent on the first or second flattened surface 54, 56 contacts the foam 12 in the receptacle 28 and allowed to cure, thus bonding the foam 12 to the stem 14.

The first and second flattened surfaces 54 and 56 are particularly designed to receive and retain the adhesive agent prior to insertion of the bonding portion 46 into the receptacle 36 and, further, to distribute the adhesive agent across appropriate portions of the foam 12 in the receptacle 36 upon inserting the bonding portion 46 therein.

In a preferred embodiment, the stem 14 is maneuvered during or immediately after insertion of the bonding portion 46 into the receptacle 36 to distribute the adhesive agent across a desired area of the foam 12 in the receptacle 36. For example, the stem 14 is twisted upon insertion of the bonding portion 46 into the receptacle 36 such that the bonding portion 46 is rotated in a spiral or semi-spiral manner within the receptacle 36, thus distributing the adhesive agent across an appropriate area of the foam 12 in the receptacle 36. In another example, the stem 14 is twisted after the bonding portion 46 is fully inserted into the receptacle 36 such that the bonding portion 46 is rotated within the receptacle 36, thus distributing the adhesive agent as desired. Of course, the stem 14 and, accordingly, the bonding portion 46 may be maneuvered in any manner (longitudingly, rotationally, etc.) before, during, or after insertion of the bonding portion 46 into the receptacle 36 to distribute the adhesive agent across the foam 12 as desired.

Maneuvering the stem 14 as described enables proper and complete distribution of the adhesive agent across the foam 12 in the receptacle 36, thus providing enhanced preparation of the respective foam and stem bonding surfaces and ensuring adequate and consistent bonding thereof.

The adhesive agent may be any material suitable for securely bonding the foam 12 and the stem 14, for example, a cyano-acrylate adhesive such as the commercially available “Loctite 416” adhesive.

The foam 12 may be composed of any suitable foam for use with the earplug 10. For example, the foam 12 may comprise a soft, pliable self-rising foam with instant recovery properties such as a polyurethane or acrylic blend foam. Other suitable foams include PVC, silicone, and nitrile, among others. A suitable foam is described, for example, in U.S. Pat. No. 5,792,998 to Gardner, Jr. et al., herein incorporated by reference. The earplug described therein is comprised of a dynamically stiff foam material having a low static stiffness, and a high dynamic stiffness. Another suitable foam is described, for example, in U.S. Pat. No. 4,158,087 to Wood, herein incorporated by reference.

While the foam 12 has been described herein as any soft, pliable self-rising foam, the invention contemplates the use of any suitable sound attenuating element for bonding with the stem 14 to form the earplug 10. For example, a plastic or rubber material may be used instead of the described foam to provide a sound attenuating element to the earplug 10. That is, in one embodiment of the earplug, a sound attenuating element of plastic, rubber, etc. includes the described skirt and receptacle and is adhesively bonded to the stem in the receptacle to form the earplug of the invention.

The stem 14 is composed of a material rigid enough to aid insertion into the wearer's ear, yet soft and pliable enough to be comfortable and safe for the wearer. The stem 14 is made of a material that is compatible with the foam 12 and which facilitates creation of a bond between the stem 14 and the foam 12. Thus, the material is preferably of a plastic material. More specifically, the stem material may be a vinyl material such as PVC having, for example, a shore durometer of 50-90. The stem 14 may be of a solid or dense construction, or may include a hollow interior, or may include one or more cavities formed therein, such as cavity 80.

The foam 12 and the stem 14 may be of identical colors. Alternatively, the stem 14 may be of a different color than the foam 12, thus clearly distinguishing the handle portion 48 of the earplug 10 for ease in inserting, removing, and otherwise identifying the earplug 10, and for allowing an observer to visually determine the extent and sufficiency of insertion of the earplug 10, e.g., visibility of the color of the skirt 20 may indicate improper insertion.

The earplug contemplated herein provides a comfortable and effective hearing protection device which may be readily manufactured in bulk quantities at a low cost with minimal labor.

The sleeve extending from the foam portion of the earplug provides increased surface area to which the stem may bonded. That is, an end of the stem as well as sides of the stem may be readily bonded to the foam. The extended sleeve enables a strong and consistent bond to be formed between the stem and the foam.

Injection molding of the stem ensures consistent manufacturing of large quantities thereof and allows for specific contouring to be applied thereto. Such contouring may be designed to provide gripping surfaces to facilitate handling of the stem and the earplug the during molding process of the stem, assembly of the plug, and throughout use of the plug by a user. Additionally, bonding surfaces, such as the flattened surfaces of the bonding portion of the stem, may be easily formed during the stem molding process to allow formation of a strong adhesive bond between the stem and the foam. Also, a certain visual presentation, such as letters, numbers, or symbols, may be readily formed on the stem during the molding thereof.

While preferred embodiments have been shown and described, various modifications and substitutions may be made thereto without departing from the spirit and scope of the invention. Accordingly, it is to be understood that the present invention has been described by way of illustrations and not limitation.

1. An earplug comprising:
a foam including an insertion portion for being inserted into an ear canal of a user and a skirt extending from the insertion portion, the skirt having a receptacle formed at an interior thereof;
a molded stem including a bonding portion received in the receptacle and a handle portion extending from the receptacle for facilitating handling of the earplug during manufacture and use of the earplug;
wherein the skirt extends around the bonding portion and wherein the bonding portion and the foam are adhesively bonded in the receptacle.

2. The earplug of claim 1, wherein the molded stem is an elongated substantially cylindrical member including opposing first and second ends, the bonding portion being disposed proximate the first end and the handle portion being disposed proximate the second end, the bonding portion being tapered in a direction toward the first end.

3. The earplug of claim 2, wherein the bonding portion includes a flattened surface for receiving a bonding agent to facilitate adhesive bonding of the bonding portion and the foam.

4. The earplug of claim 3, wherein the flattened surface includes a width tapered in a direction toward the first end.

5. The earplug of claim 3, wherein the molded stem is formed in an injection molding process and the bonding agent is a cyano-acrylate adhesive.

6. The earplug of claim 2, wherein the handle portion is substantially a curvilinear elongated member including first and second opposite sides and third and fourth opposite sides, the first and second sides each having a width greater than a width of each of the third and fourth sides.

7. The earplug of claim 6, wherein the third and fourth sides include each include a recessed portion.

8. The earplug of claim 7, wherein the recessed portion provides a gripping surface to facilitate said handling of the earplug during manufacture and use of the earplug.

9. The earplug of claim 8, wherein the recessed portion comprises a visual display area including characters or symbols for visual observation by an observer.

10. The earplug of claim 1, wherein the stem includes a cavity formed at an interior of the stem and an opening at an end of the stem to expose the cavity to an exterior of the stem.

11. The earplug of claim 10, wherein a cord is inserted through the opening into the cavity and fixed to the stem.

12. The earplug of claim 1, wherein the stem further includes a collar disposed intermediate the bonding portion and the handle portion, the collar being substantially cylindrical in shape and extending circumferentially from the stem.

13. The earplug of claim 12, wherein the collar has a cross-sectional area greater than a cross-sectional area of the bonding portion.

14. The earplug of claim 13, wherein the cross-sectional area of the collar is greater than a cross-sectional area of the handle portion.

15. The earplug of claim 1, wherein the insertion portion has a hemispherical shape to facilitate said insertion of the earplug into the ear canal of the wearer.

16. The earplug of claim 2, wherein the skirt has a cylindrical shape and extends to a point substantially equidistant from the first and second ends and wherein the receptacle has a shape corresponding to a shape of the bonding portion.

17. The earplug of claim 16, wherein the receptacle has a substantially cylindrical shape.

18. The earplug of claim 1, wherein the foam further includes ribs extending from the insertion portion to the skirt formed during formation of the foam to allow a venting of the foam.

19. The earplug of claim 18, wherein the skirt has a substantially cylindrical shape and the ribs include first, second, third, and fourth ribs extending radially from the cylindrical skirt, wherein the first, second, third, and fourth ribs are spaced equally about an exterior of the cylindrical skirt.

20. An earplug, comprising:
a stem including a first end, a second end opposite the first end, a bonding portion disposed proximate the first end, and a handle portion disposed proximate the second end, the bonding portion being tapered in a direction toward the first end and having a flattened surface for receiving a bonding agent, the handle portion having contouring for facilitating handling of the earplug during manufacture and use of the earplug; and
a sound attenuating element;
wherein the bonding portion is bonded by the bonding agent to the sound attenuating element and wherein the handle portion of the stem extends from the sound attenuating element.

21. The earplug of claim 20, wherein the sound attenuating element is a hemispherical foam including a receptacle for receiving the bonding portion.

22. The earplug of claim 21, wherein the receptacle is formed at an interior of a skirt which extends from the foam, the skirt covering the bonding portion.

23. An earplug stem, comprising:
a first end;
a second end disposed opposite the first end;
a bonding portion disposed proximate the first end including a flattened surface, the bonding portion and the flattened surface being tapered in a direction toward the first end; and
a handle portion including contouring for facilitating handling of the earplug stem during manufacture and use of the earplug stem.

24. The earplug stem of claim 23, wherein the contouring includes a recessed portion formed in the handle portion, the recessed portion comprising a gripping surface to facilitate said handling of the earplug stem during manufacture and use thereof.

25. The earplug of claim 24, wherein the recessed portion further comprises a visual display area including characters of symbols for visual observation by an observer.

26. The earplug of claim 23, further comprising a collar disposed intermediate the bonding portion and the handle portion, the collar having a substantially cylindrical shape and having a cross-sectional area larger than a cross-sectional area of the bonding portion and larger than a cross-sectional area of the handle portion.

27. A method of manufacturing earplugs, comprising:
molding a foam to include a rounded portion and a skirt extending from the rounded portion;
forming a receptacle at an interior of the skirt;
molding a stem to include a bonding portion having a flattened surface and a handle portion having contouring to facilitate gripping the stem;
applying a bonding agent to the flattened surface;
inserting the bonding portion into the receptacle; and
curing the bonding agent to adhesively bond the bonding portion and the foam in the receptacle.

28. The method of claim 27, further comprising maneuvering the bonding portion in the receptacle to spread the bonding agent on an interior surface of the receptacle.

29. The method of claim 28, wherein said maneuvering comprises rotating the bonding portion when inserting the bonding portion into the receptacle.

30. The method of claim 27, wherein said molding the stem comprises an injection molding process.

31. The method of claim 27, wherein said molding the stem further comprises forming a visual display area on the handle portion including characters or symbols for visual observation by an observer.
